(19) United States
(12) Patent Application Publication
(10) Pub. No.: US 2025/0288646 A1
(43) Pub. Date: Sep. 18, 2025

(54) PHARMACEUTICAL COMPOSITION COMPRISING CDON-IG2 DOMAIN PROTEIN FOR TREATING VASCULAR CALCIFICATION

(71) Applicant: ANIMUSCURE INC., Suwon-si, Gyeonggi-do (KR)

(72) Inventors: Sangjin LEE, Seoul (KR); Byeong-Yun ANH, Suwon-si (KR); Young-Eun LEEM, Suwon-si (KR)

(73) Assignee: ANIMUSCURE INC., Suwon-si, Gyeonggi-do (KR)

(21) Appl. No.: 18/860,865

(22) PCT Filed: Apr. 24, 2023
(86) PCT No.: PCT/KR2023/005555

§ 371 Date: Jan. 24, 2025

(30) Foreign Application Priority Data
Apr. 28, 2022 (KR) 10-2022-0052520

(51) Int. Cl. A61K 38/17; A61P 9/14; G01N 33/68

(52) U.S. Cl. CPC A61K 38/1774; A61P 9/14; G01N 33/6893; G01N 2333/70503; G01N 2800/32; G01N 2800/50

(57) ABSTRACT

The present invention relates to a composition for preventing, ameliorating, or treating vascular calcification and a vascular disease associated therewith, which includes a Cdon-Ig2 domain or a peptide derived therefrom, and a method of providing information on the diagnosis of a disease associated with vascular calcification through the confirmation of the expression level of the peptide.

### TECHNICAL FIELD

The present invention relates to the use of a Cdon-Ig2 domain or a peptide derived therefrom for preventing, ameliorating, or treating vascular calcification and a vascular disease associated therewith, and a method of providing information related to vascular calcification through expression of the peptide.

### BACKGROUND ART

Vascular calcification, a disease in which calcium accumulates in blood vessels and hardens, is often observed in patients with arteriosclerosis and may reduce the elasticity of the aorta and arteries. Vascular calcification may be classified into arterial intimal calcification (AIC) and arterial medial calcification (AMC) depending on the region where it occurs. In particular, AIC occurs in relation to atherosclerosis, and AMC is commonly observed in patients with type 2 diabetes and end-stage renal disease, and this type of calcification is also referred to as Mönckeberg medial sclerosis (MMS). Vascular calcification causes vascular sclerosis, systolic hypertension and blood pressure fluctuations, which may eventually lead to cardiac hypertrophy, myocardial ischemia, peripheral arterial ischemia, and congestive heart failure.

Cell adhesion molecule (CAM)-related/downregulated by oncogenes (Cdon) is a member of the immunoglobulin/fibronectin type III superfamily of cell adhesion molecules, and it has been confirmed through previous studies that Cdon plays an important role in the development of the forebrain and skeletal muscle through the regulation of Shh, Wnt, and N-cadherin/cell adhesion signals. In addition to activating Shh signaling, Cdon inhibits Wnt signaling through interactions with the Lrp6 co-receptor, thereby promoting ventral neuronal development in early forebrain development. In addition, the inhibitory activity of Cdon on Wnt signaling has been demonstrated in the prevention of cardiac remodeling and fibrosis. In other words, it has been confirmed through various studies that Cdon has the activity of inhibiting Wnt signaling, or that when Cdon is overexpressed, Wnt signaling is inhibited, which may affect a series of subsequent processes such as osteogenesis, neuronal development, or cardiac remodeling.

Patients with chronic kidney disease (CKD) exhibit severe arterial calcification and have a high incidence of cardiovascular disease, and the high blood phosphate concentrations in patients with mineral and bone diseases may induce vascular calcification by activating Wnt/β-catenin signaling. Klotho mutant (kl/kl) mice exhibit severe tissue calcium deposition including AIC. Klotho is known to bind to several Wnt ligands and inhibit Wnt signaling activation, and thus Wnt signaling is activated in kl/kl mice. Klotho is mainly produced in the kidneys and the secreted form is released into the blood, but patients with CKD show reduced klotho production. In other words, when klotho production is reduced, the Wnt signaling pathway may be activated, and in this case, research is needed to determine whether vascular calcification may be treated through the regulation of the Wnt signaling pathway.

Accordingly, the present inventors conducted research on a substance capable of treating vascular calcification among various factors involved in the Wnt signaling pathway, thereby completing the present invention.

### DISCLOSURE

### Technical Problem

The present inventors discovered that among factors involved in the Wnt/β-catenin signaling pathway, the Cdon-Ig2 domain is associated with vascular calcification and may treat it, and completed the present invention.

Therefore, an object of the present invention is to provide a composition for preventing, ameliorating, or treating vascular calcification or a vascular disease, including a Cdon-Ig2 domain or a peptide derived therefrom.

Another object of the present invention is to provide a composition for preventing, ameliorating, or treating vascular calcification or a vascular disease, including a vector including a polynucleotide encoding a Cdon-Ig2 domain or a peptide derived therefrom, and a cell transfected with the vector.

Still another object of the present invention is to provide a method of providing information on vascular calcification or a vascular disease, including: 1) measuring the expression level of a Cdon-Ig2 domain or a peptide derived therefrom in a sample derived from a subject as an experimental group;
2) comparing the expression level of the Cdon-Ig2 domain or the peptide derived therefrom with the expression level of a Cdon-Ig2 domain or a peptide derived therefrom in a sample derived from a normal subject as a control group; and
3) determining that the risk of developing vascular calcification or a vascular disease associated therewith is high when the expression level of the Cdon-Ig2 domain or the peptide derived therefrom is lower compared to the control group.

### Technical Solution

To achieve the above-described object, the present invention provides a composition for preventing, ameliorating, or treating vascular calcification or a vascular disease, including a Cdon-Ig2 domain or a peptide derived therefrom.

To achieve another object of the present invention, the present invention provides a composition for preventing, ameliorating, or treating vascular calcification or a vascular disease, including a vector including a polynucleotide encoding a Cdon-Ig2 domain or a peptide derived therefrom, and a cell transfected with the vector.

To achieve still another object of the present invention, the present invention provides a method of providing information on vascular calcification or a vascular disease, including:
1) measuring the expression level of a Cdon-Ig2 domain or a peptide derived therefrom in a sample derived from a subject as an experimental group;
2) comparing the expression level of the Cdon-Ig2 domain or the peptide derived therefrom with the expression level of a Cdon-Ig2 domain or a peptide derived therefrom in a sample derived from a normal subject as a control group; and
3) determining that the risk of developing vascular calcification or a vascular disease associated therewith is high when the expression level of the Cdon-Ig2 domain or the peptide derived therefrom is lower compared to the control group.

Hereinafter, the present invention will be described in detail.

The present invention relates to a pharmaceutical composition for preventing, ameliorating, or treating vascular calcification or a vascular disease associated therewith, including a Cdon-Ig2 domain or a peptide derived therefrom.

Cdon (also referred to as Cdo), a cell surface protein, is a cell surface receptor of the immunoglobulin/fibronectin type III repeat family involved in muscle differentiation. Cdon forms a complex with N-cadherin at a contact site between skeletal myoblasts, and this interaction is known to have a significant effect on Cdon's function of promoting muscle cell differentiation. Nothing is known about the mechanism by which Cdon is expressed in various motor nerves or neuromuscular junctions. In addition, the present inventors confirmed through previous studies that Cdon binds to Lrp6 and inhibits the Wnt signaling pathway. In the present invention, it was confirmed that the Ig2 domain of Cdon has an effect of inhibiting vascular calcification, and through this, it was confirmed that the Ig2 domain of Cdon may be utilized for the prevention, amelioration, or treatment of a disease associated with vascular calcification.

The Cdon-Ig2 domain of the present invention specifically includes a peptide or a fragment thereof derived from mammalian Cdo or Cdon consisting of an amino acid sequence of SEQ ID NO: 1 or SEQ ID NO: 2, and more specifically, it may include a peptide consisting of an amino acid sequence of SEQ ID NO: 3 or SEQ ID NO: 4. In addition, the Cdon-Ig2 domain may be a peptide including an artificially synthesized sequence, and specifically, it may be a peptide including an amino acid sequence of SEQ ID NO: 5 to SEQ ID NO: 11, and more specifically, it may be a peptide including an amino acid sequence of SEQ ID NO: 7 to SEQ ID NO: 10.

As another aspect of the present invention, the present invention relates to a composition for preventing, ameliorating, or treating vascular calcification or a vascular disease associated therewith, using a vector including a polynucleotide encoding the Cdon-Ig2 domain or a peptide derived therefrom and a cell transfected with the vector.

The vector including the polynucleotide of the Cdon-Ig2 domain or the peptide derived therefrom is not limited in type, and may be used without limitation as long as it is capable of expressing the Cdon-Ig2 domain or the peptide derived therefrom in vivo or in vitro. Specifically, it may be a vector including linear DNA, plasmid DNA, or a recombinant viral vector expressed in human or animal cells, and the recombinant viral vector preferably includes a recombinant retrovirus vector, a recombinant adenovirus vector, a recombinant adeno-associated virus (AAV) vector, a recombinant herpes simplex virus vector, or a recombinant lentivirus vector, but is not limited thereto.

In addition, the cell transfected with the vector including a polynucleotide encoding the Cdon-Ig2 domain or the peptide derived therefrom may be a vascular cell, which may be selected from a vascular muscle cell, a vascular smooth muscle cell, a vascular endothelial cell, an angioblast, or a vascular stem cell.

The term ‘vascular calcification’ as used herein refers to a phenomenon that occurs when calcium in the body is not normally utilized and is deposited in the vascular wall or organ cells together with waste products. The vascular calcification can be classified into arterial intimal calcification (AIC) and arterial medial calcification (AMC) depending on the region where it occurs, and the vascular calcification herein includes both of these. In addition, associated symptoms or vascular diseases that may be caused by such vascular calcification may include myocardial infarction, angiosclerosis (arteriosclerosis), hypertension, ischemic heart disease, perfusion disorders, cardiac hypertrophy, left ventricular hypertrophy, myocardial ischemia, peripheral arterial ischemia, and heart failure, and in addition, vascular diseases caused by vascular calcification may be included without limitation. The Cdon-Ig2 domain or the peptide derived therefrom of the present invention may exhibit a therapeutic effect on the above-described diseases through the amelioration of vascular calcification.

In other words, in the present invention, the preventive, therapeutic, or ameliorating effect of the Cdon-Ig2 domain on the vascular disease is due to the reduction of vascular calcification. In one embodiment of the present invention, it was confirmed that the Cdon-Ig2 domain included in the Cdon affects the expression of a regulatory factor related to vascular calcification. As confirmed in one embodiment of the present invention, when vascular calcification was artificially induced using mouse vascular smooth muscle cells (A7r5 cells), the expression levels of Runx2 and alkaline phosphatase (ALPL), which are osteocyte differentiation markers, relatively increased, but the expression level of Cdon drastically decreased. In addition, it was confirmed that even when the expression of Cdon was knocked out, the expression levels of Runx2 and ALPL relatively increased and vascular calcification increased. From this, it was confirmed that Cdon is closely related to vascular calcification.

The term “prevention” as used herein refers to any action that may inhibit or delay the onset of vascular calcification by administering the pharmaceutical composition according to the present invention.

The term “treatment” as used herein refers to any action that ameliorates symptoms or is beneficial by administering the pharmaceutical composition according to the present invention.

The pharmaceutical composition of the present invention may be formulated and used in the form of oral dosage forms such as powder, granules, tablets, capsules, suspensions, emulsions, syrups, and aerosols, external preparations, suppositories, and sterile injection solutions according to conventional methods, and may further include carriers or excipients necessary for the formulation. Pharmaceutically acceptable carriers, excipients, and diluents that may be additionally included along with the active ingredient include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, magnesium stearate, and mineral oil. When formulated, the preparation is prepared using commonly used diluents or excipients such as fillers, bulking agents, binders, wetting agents, disintegrants, and surfactants.

For example, solid preparations for oral administration include tablets, pills, powder, granules, capsules, and the like, and these solid preparations are prepared by mixing the extract or compound with at least one excipient such as cotton, starch, calcium carbonate, sucrose or lactose, gelatin, and the like. In addition to simple excipients, lubricants such as magnesium stearate and talc are also used. Liquid preparations for oral administration include suspensions, oral solutions, emulsions, syrups, and the like, and in addition to commonly used simple diluents such as water and liquid paraffin, various excipients such as wetting agents, sweeteners, flavoring agents, and preservatives may be included.

Preparations for parenteral administration include sterile aqueous solutions, non-aqueous solutions, suspensions, emulsions, lyophilized preparations, and suppositories. Non-aqueous solutions and suspensions may include propylene glycol, polyethylene glycol, vegetable oils such as olive oil, and injectable esters such as ethyl oleate. As a base for suppositories, Witepsol, Macrogol, Tween 61, cacao oil, laurel oil, glycerol, gelatin, or the like may be used.

The pharmaceutical composition of the present invention may be administered orally or parenterally (intravenously, subcutaneously, intraperitoneally, or topically) depending on the intended method, and the dosage varies depending on the patient's conditions and weight, the severity of the disease, the drug form, and the administration route and time, and may be selected in an appropriate form by one of ordinary skill in the art.

The pharmaceutical composition according to the present invention is administered in a pharmaceutically effective amount. The term “pharmaceutically effective amount” as used herein refers to an amount that is sufficient to treat a disease and a reasonable amount applicable to medical treatment, and the amount may be determined based on factors including the type and severity of a patient's disease, drug activity, sensitivity to the drug, administration time, administration route and excretion rate, treatment duration, and concurrently used drugs, and other factors. The pharmaceutical composition according to the present invention may be administered as an individual therapeutic agent or in combination with another therapeutic agent, and it may be administered sequentially or simultaneously with conventional therapeutic agents. The dosage may be determined at a level that may minimize side effects by considering all of the above factors, and this may be easily determined by one of ordinary skill in the art to which the present invention pertains. Specifically, the dosage of the pharmaceutical composition may vary depending on the patient's age, weight, severity, sex, and the like, and the pharmaceutical composition may generally be administered in an amount of 0.001 to 150 mg, more preferably 0.01 to 100 mg, per 1 kg of body weight once to three times a day every day or every other day. However, this is exemplary, and the dosage may be set differently as needed.

In addition, as one embodiment of the present invention, the present invention relates to a health food composition for preventing or ameliorating vascular calcification or a vascular disease associated therewith, including a Cdon-Ig2 domain or a peptide derived therefrom. The “health functional food” refers to a food that is manufactured and processed using raw materials or ingredients having functionality useful to the human body in accordance with Health Functional Food Act No. 6727 and ingested for the purpose of regulating nutrients for the structure and functions of the human body or obtaining useful effects for healthcare purposes such as physiological actions.

The food or health functional food of the present invention may be manufactured and processed into pharmaceutical dosage forms such as powder, granules, tablets, capsules, pills, suspensions, emulsions, syrups, or health functional foods such as tea bags, infusions, beverages, candy, jelly, and gum for the purpose of preventing and ameliorating vascular calcification and associated diseases.

The food or health functional food composition of the present invention may be used as a food additive, and may be manufactured into a product alone or in combination with other ingredients. In addition, nutrients, vitamins, electrolytes, flavoring agents, coloring agents, and enhancing agents, pectic acid and salts thereof, alginic acid and salts thereof, organic acids, protective colloid thickeners, pH regulators, stabilizers, preservatives, glycerin, alcohols, carbonating agents used in carbonated beverages, and the like may be included. The above-described ingredients may be used alone or in combination, and may be used in combination in appropriate amounts.

As another aspect of the present invention, the present invention provides a method of providing information on vascular calcification or a vascular disease, including:
1) measuring the expression level of a Cdon-Ig2 domain or a peptide derived therefrom in a sample derived from a subject as an experimental group;
2) comparing the expression level of the Cdon-Ig2 domain or the peptide derived therefrom with the expression level of a Cdon-Ig2 domain or a peptide derived therefrom in a sample derived from a normal subject as a control group; and
3) determining that the risk of developing vascular calcification or a vascular disease associated therewith is high when the expression level of the Cdon-Ig2 domain or the peptide derived therefrom is lower compared to the control group.

In one embodiment of the present invention, it was confirmed that when the expression of Cdon was inhibited, vascular calcification could be induced, and conversely, when the expression of Cdon was induced, vascular calcification was reduced. Therefore, by confirming the expression of Cdon, the onset of vascular calcification or a disease associated therewith may be diagnosed. In the detection method for providing the above diagnostic information, the expression level of the Cdon-Ig2 domain or the peptide derived therefrom in step 1) is preferably detected by any one method selected from the group consisting of Western blotting, real-time polymerase chain reaction (qRT-PCR), enzyme-linked immunosorbent assay (ELISA), (immunohistochemical staining, immunoprecipitation, and immunofluorescence, but is not limited thereto.

Therefore, the Cdon-Ig2 domain or the peptide derived therefrom of the present invention reduces the expression of factors associated with calcification, thereby alleviating calcification in vascular cells and a vascular disease associated therewith, and the onset of vascular calcification or a vascular disease may be diagnosed through the expression of the factors associated with calcification. Therefore, the Cdon-Ig2 domain or the peptide derived therefrom may be utilized in the treatment and diagnosis of vascular calcification and a vascular disease associated therewith.

### Advantageous Effects

The present invention relates to the use of a Cdon-Ig2 domain or a peptide derived therefrom for the prevention, amelioration, or treatment of vascular calcification and a vascular disease associated therewith. Since the Cdon-Ig2 domain has an effect of alleviating vascular calcification, it can be used as a pharmaceutical or health functional food composition for vascular calcification and associated diseases, and can be used to provide information related to the diagnosis of vascular calcification and vascular diseases.

### DESCRIPTION OF DRAWINGS

FIG. 1 shows a schematic diagram of the sequence of the Cdon-Ig2 peptide artificially synthesized using a rat Cdon-Ig2 domain and the three-dimensional structure thereof.

FIG. 2 shows a diagram confirming that calcification was induced after the vascular smooth muscle cells (VSMC) (A7r5 cells) were cultured in a vascular calcification medium (CM) to induce vascular calcification.

FIG. 3 shows a diagram confirming that the expression of bone formation markers Runx2 and ALPL was increased in VSMCs in which calcification was induced, by performing a real-time polymerase chain reaction (the data is expressed as ±standard error of measure (SEM) analyzed by Student's t-test, **p<0.01, ***p<0.005).

FIG. 4 shows a diagram confirming that the expression level of Cdon was decreased compared to the increase in bone formation markers in the VSMCs in which calcification was induced (the data is expressed as mean±SEM analyzed by Student's t-test, *p<0.05)

FIG. 5A shows immunostaining images of Cdon and αSMA in the aorta in which calcification was induced by injecting vitamin D3 (VD3) (bars: 100 μm (first) and 50 μm (second)). FIG. 5B shows the expression level of Cdon quantified by normalizing it to the intensity of αSMA based on the fluorescence intensity of the measured immunostaining image (n=4, the data is expressed as ±SEM analyzed by Student's t-test, *p<0.05). FIG. 5C shows the relative mRNA expression level of Cdon (the data is expressed as ±SEM analyzed by Student's t-test, **p<0.01).

FIG. 6 shows the results of inducing calcification in VSMCs in which Cdon was knocked out, by culturing the cells in a CM, as confirmed by using alizarin red staining (scale bar: 100 μm).

FIG. 7 shows the results of measuring the relative mRNA levels of bone formation markers Runx2 and ALPL in VSMCs (A7r5 cells) in which Cdon was knocked out and then calcification was induced (the data is expressed as ±SEM analyzed by Student's t-test, ** p<0.01, ***p<0.005).

FIG. 8 shows the results of an immunoblot analysis of VSMCs (A7r5 cells) infected with a lentivirus containing shCdon with Cdon knocked out.

FIG. 9 shows the area where calcification occurred and the expression level of Runx2, a bone formation marker, confirmed by Von Kossa staining and immunofluorescence staining, respectively, after VD3 was injected into the VSMCs of the mice (cKO) in which Cdon was knocked out (scale bars: 100 μm (top), 40 μm (bottom)).

FIG. 10 shows the results of comparing the changes in pulse wave velocity (PWV) after vascular sclerosis was induced in the mouse model in which Cdon was knocked out and a normal group (the data is expressed as ±SEM analyzed by Student's t-test, *p<0.05, *** p<0.005).

FIG. 11 shows the results of an immunoblotting analysis of b-catenin and Runx2 expression in VSMCs expressing Cdon (WT) or Cdon (ΔIg2) with the Ig2 domain knocked out.

FIG. 12 shows the relative expression level of Axin2, a key factor in Wnt signaling, in Cdon- or ΔIg2-expressing VSMCs in which calcification was induced by a CM (the data is expressed as ±SEM analyzed by one-way analysis of variance (ANOVA), n.s.=not significant, *p<0.05, **p<0.01).

FIG. 13 shows alizarin red staining images and the quantification graph of Cdon- or ΔIg2-expressing VSMCs in which calcification was induced by a CM (scale bar: 100 μm).

FIG. 14 shows a diagram illustrating the Cdon-Fc protein (protein in which the entire ectodomain of Cdon is fused to Fc) and the Ig2-Fc protein (protein in which the Cdon-Ig2 domain is fused to Fc).

FIG. 15 shows the results of an immunoblotting analysis of the expression of b-catenin and Runx2 in VSMCs cultured in a CM containing the purified protein of FIG. 14.

FIG. 16 shows the alizarin red staining images and a quantification graph of the VSMCs cultured in a CM treated with Cdon-Fc or Ig2-Fc (the data is expressed as ±SEM analyzed by one-way ANOVA test, ***p<0.005).

FIG. 17 shows a diagram illustrating the differences in mRNA expression levels of Axin2, a Wnt signaling marker, and Runx2 and ALPL, bone formation markers, in VSMCs cultured in a CM treated with Cdon-Fc and Ig2-Fc (the data is expressed as ±SEM analyzed by Student's t-test, *p<0.05, ***p<0.005).

### MODES OF THE INVENTION

Hereinafter, examples will be described in detail to specifically explain the present invention. However, the examples according to the present invention may be modified into various different forms, and the scope of the present invention is not to be construed as being limited to the examples described below. The examples of the present invention are provided to more completely explain the present invention to a person having average knowledge in the art.

### Example 1. Preparation of Cdon-Ig2 Domain and Peptide

### 1-1. Isolation and Purification of Cdon-Ig2 Domain Derived from Human or Rat

The Cdon of the Present Invention is Derived from UNIPROT Q4KMG0-1 (human) or NCBI NP_059054.2 (rat), which may be represented by the amino acid sequences of SEQ ID NO: 1 and SEQ ID NO: 2, respectively. In addition, a part corresponding to the Cdon-Ig2 domain of the present invention is included in the entire Cdon sequence, and includes the amino acid sequences of SEQ ID NO: 3 (human-derived) and SEQ ID NO: 4 (rat-derived), respectively.

In addition, to produce each of Cdon-Fc and Ig2-Fc fusion proteins, the recombinant protein of Cdon was combined with the Fc region of human IgG gamma and transfected into HEK293T cells (Cdon-Fc: fusion protein of the entire ectodomain of Cdon and Fc; Cdon-Ig2-Fc: fusion protein of the Ig2 domain of Cdon and Fc). After transfection for 72 hours, the HEK293T cells were centrifuged at 2,000×g for 10 minutes, and the supernatant was filtered through a 0.45 μm filter and allowed to react with protein A agarose for one hour at room temperature. The protein bound to protein A agarose was eluted with 0.1 M citric acid (pH 3.0), and the eluted protein was concentrated using the Amicon Ultra-4 Centrifugal Filter (MWCO 3K, Millipore). The purity and integrity of the concentrated protein were confirmed by sodium dodecyl-sulfate polyacrylamide gel electrophoresis (SDS-PAGE).

### 1-2. Isolation and Purification of Cdon-Ig2 Domains Derived from Human or Rat

A Cdon-Ig2 domain was artificially synthesized based on a rat-derived sequence (SEQ ID NO: 12). The synthesized domain included the amino acid sequences of SEQ ID NOs: 5 to 11, and the sequence information is as shown in Table 1 below. In addition, it was confirmed that the synthesized Cdon-Ig2 domain included the sequences of SEQ ID NOs: 7 to 10 outside the three-dimensional structure. The three-dimensional structure of the synthesized protein is illustrated in FIG. 1.

TABLE 1

| Position | Sequence | SEQ ID NO. |
|---|---|---|
| Cdon-ig2 Ig strand A (120-123) | FDSS | SEQ ID NO: 5 |
| Cdon-ig2 Ig strand B (133-144) | KNTGFIGCRVPE | SEQ ID NO: 6 |
| Cdon-ig2 Ig strand C (149-155) | AEVRYKI | SEQ ID NO: 7 |
| Cdon-ig2 Ig strand D (157-160) | GKWL | SEQ ID NO: 8 |
| Cdon-ig2 Ig strand E (167-171) | YIILP | SEQ ID NO: 9 |
| Cdon-ig2 Ig strand F (173-179) | GNLQILN | SEQ ID NO: 10 |
| Cdon-ig2 Ig strand G (185-194) | KGSYKCAAYN | SEQ ID NO: 11 |

### Example 2. Cell Isolation and Culture

The A7r5 cell line (ATCC, CRL-1444) was obtained as the mouse vascular smooth muscle cells (VSMCs) for inducing vascular calcification, and the experiment was conducted with the cell line. In addition, for a Cdon knock-out experiment, VSMCs were directly isolated from a mouse in which Cdon was knocked out. Blood vessels were isolated from six to eight week-old rats, placed in a collagenase solution (0.2% collagenase type I, elastase II, bovine serum albumin (BSA), trypsin inhibitor, 15 mM HEPES, Ham's F12 medium), and cultured with shaking at 37° C. for 30 minutes. After the culture solution settled, the endothelial cells were scraped off, and the outer membrane was peeled off and then finely chopped in a dissection culture solution (penicillin, streptomycin, Ham's F12 medium). The dissection culture solution was discarded, and then the cells were placed in a collagenase solution again, and cultured with shaking at 37° C. for 90 minutes. After stopping the collagenase reaction by adding Dulbecco's modified eagle medium (DMEM) containing 10% (v/v) fetal bovine serum (FBS) to the above cell supernatant, the cells were cultured in an incubator at 37° C., and the next day, the medium was replaced with a 10% FBS/DMEM medium and the cells were cultured.

### Experimental Example 1. Effect of Calcification Induction in Vascular Cells on Cdon Expression

The following experiment was performed to confirm the change in the expression level of Cdon when vascular calcification was induced in VSMCs.

To induce calcification in A7r5 cells, which are VSMCs, the cells were cultured in a calcification medium (CM) containing 10 mM β-glycerophosphate, 1 mM insulin, 8 mM CaCl₂, 100 nM dexamethasone, and 50 mg/ml ascorbic acid.

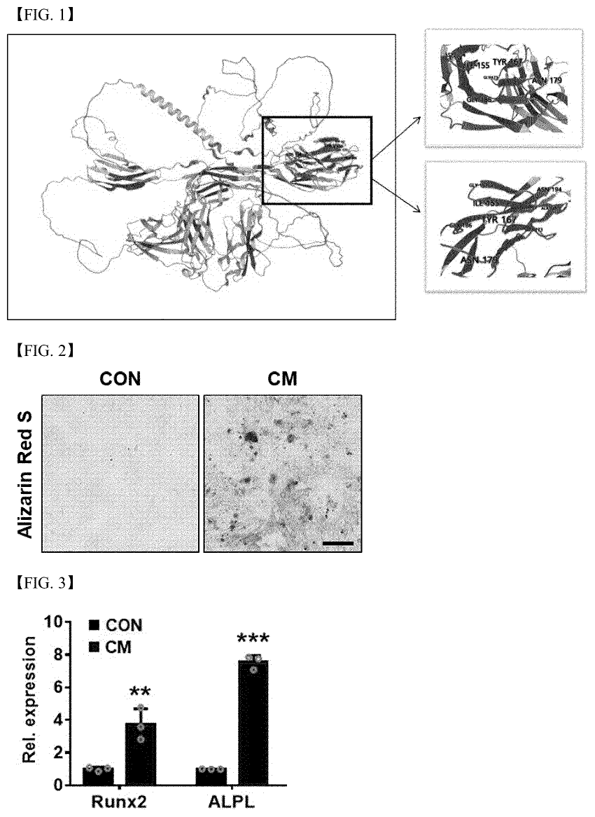

The cultured VSMCs were stained with an alizarin red solution to confirm the induction of calcium production, that is, calcification. As shown in FIG. 2, it was confirmed that calcium was deposited and thus calcification was induced in the VSMCs cultured in the CM medium, and it was confirmed that the mRNA expression of Runx2 and ALPL, which are bone differentiation-related markers, was increased (FIG. 3). However, the expression of Cdon was relatively decreased compared to the control group (FIG. 4). These results show that the expression level of Cdon is decreased when vascular calcification is induced.

In addition, an experiment was performed to confirm the change in the expression levels of Cdon and αSMA when calcification is induced in actual blood vessels. Calcification was induced by injecting vitamin D3 into the mouse aorta, and the relative expression levels of Cdon/αSMA in the aorta was confirmed through immunostaining.

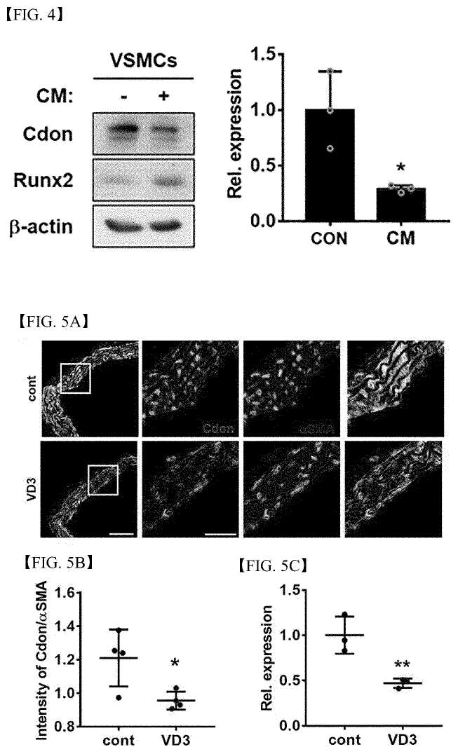

As a result, as shown in FIG. 5, it was confirmed that in the normal aorta (Cont), Cdon was highly expressed in the medial smooth muscle area and the endothelial cell area, but when calcification was induced by injecting VD3, the expression of the Cdon protein and transcript in the aorta was drastically reduced compared to the control group.

### Experimental Example 2. Aggravation of Vascular Calcification and Vascular Sclerosis Due to Cdon Knock-Out

### 2-1) Induction of Calcification in VSMCs

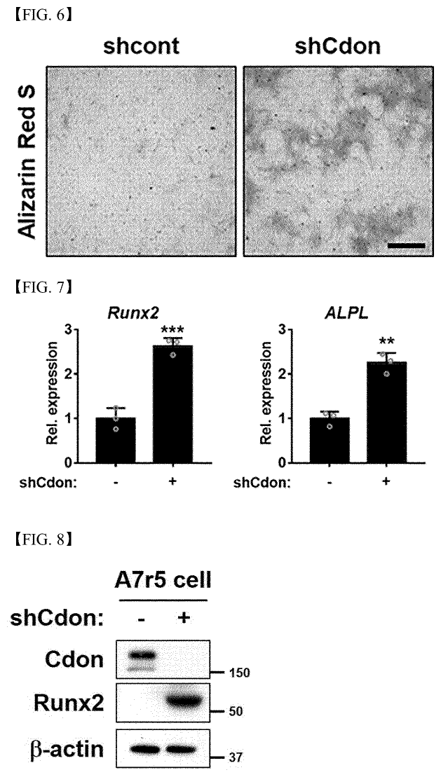

To knock out Cdon expression in the primary cultured VSMCs cultured as in Example 2, shCdon was used. Thereafter, calcification was induced in the VSMCs in which Cdon was knocked out. The cells were cultured for 14 days in a CM containing an alpha minimum essential medium (αMEM), 2 mM sodium orthophosphate, and 10 mM glycerophosphate, and half of the culture medium was replaced with a fresh medium every three days. As a result, it was confirmed that calcification was induced in the VSMCs in which Cdon was knocked out using shCdon, as shown in FIG. 6. In addition, it was confirmed that the expression levels of Runx2 and ALPL, which are bone differentiation markers that allow confirmation of calcification, were significantly increased in the Cdon knock-out cells compared to the control group (FIGS. 7 and 8).

### 2-2) Induction of Aortic Sclerosis by Vitamin D3

An experiment was performed to confirm the effect of Cdon knock-out in an animal model in which aortic sclerosis was induced. Tamoxifen (Tmx) was injected into 10-week-old mice to induce Cdon knock-out in the vascular smooth muscle of the mice, and 12 weeks later, vitamin D3 (VD3) was administered three times at 3-day intervals at a dose of 13 mg/kg to normal mice and Cdon conditional knock-out (cKO) mice to induce vascular sclerosis. Thereafter, the pulse wave velocity (PWV) and the expression levels of bone differentiation markers in the aortic vessels of each mouse model were measured and compared.

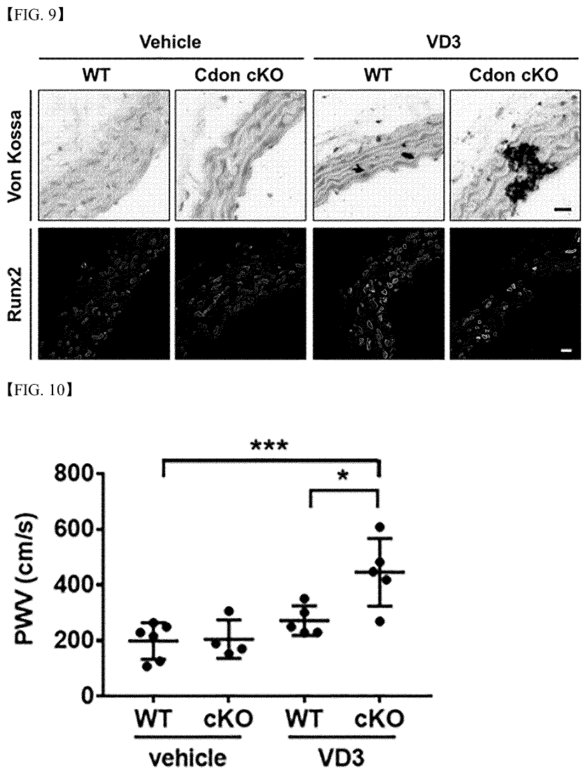

As a result, as shown in FIG. 9, when Cdon was knocked out, the calcification area induced by VD3 was significantly increased, and the expression of Runx2, a bone formation marker related to calcification, was also drastically increased. In addition, as confirmed in FIG. 10, when vascular sclerosis was induced, the PWV increased to some extent, and the increase in the PWV was more prominent in the Cdon knock-out mice.

### Experimental Example 3. Effect of Cdon-Ig2 Knock-Out on Vascular Calcification

As in Example 2-1, Cdon WT or Ig2 domain knock-out mutant Cdon (ΔIg2) was transfected into the A7r5 cells, which are primary cultured VSMCs, and then the cells were treated with a CM to induce calcification.

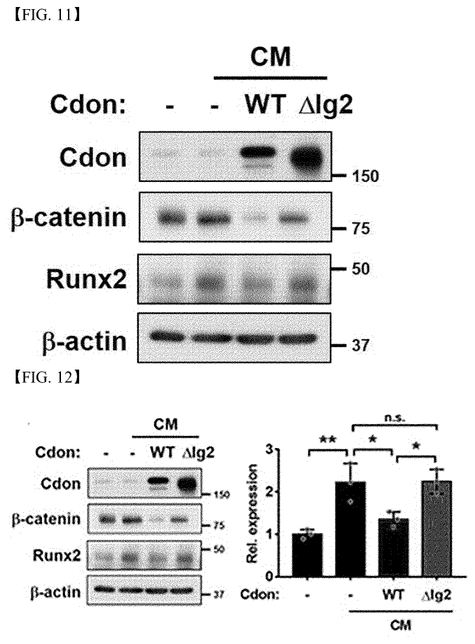

As a result, as shown in FIG. 11, when Cdon WT was transfected, the expression of β-catenin and Runx2 decreased, but when the Ig2 domain knock-out mutant was transfected, β-catenin and Runx2 were expressed at almost the same levels as the control group. In addition, as shown in FIG. 12, the expression of Axin2, a key factor in the Wnt signaling pathway, decreased when Cdon WT was transfected, but expression was maintained when the Ig2 domain knock-out mutant was transfected. In other words, it was confirmed that when calcification was induced with a CM, calcification was significantly reduced when Cdon WT was transfected, but calcification was not inhibited when the Ig2 domain knock-out mutant was transfected (FIG. 13).

### Experimental Example 4. Calcification-Reducing Effect of Cdon-Ig2 Expression

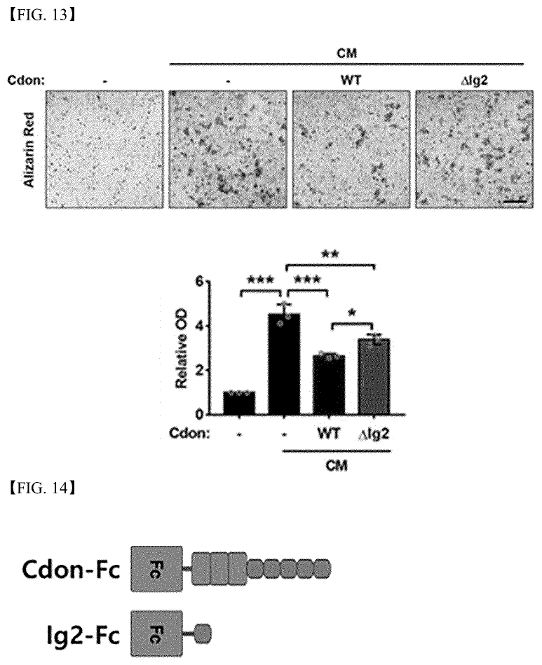

To confirm the treatment effect of Cdon-Ig2 on vascular calcification, as in Example 2-1, each of Cdon-Fc and Ig2-Fc was transfected into the A7r5 cells, which are primary cultured VSMCs, (FIG. 14), and then the cells were cultured in a CM in the same manner as Experimental Example 2 to induce calcification and confirm the effect on calcification.

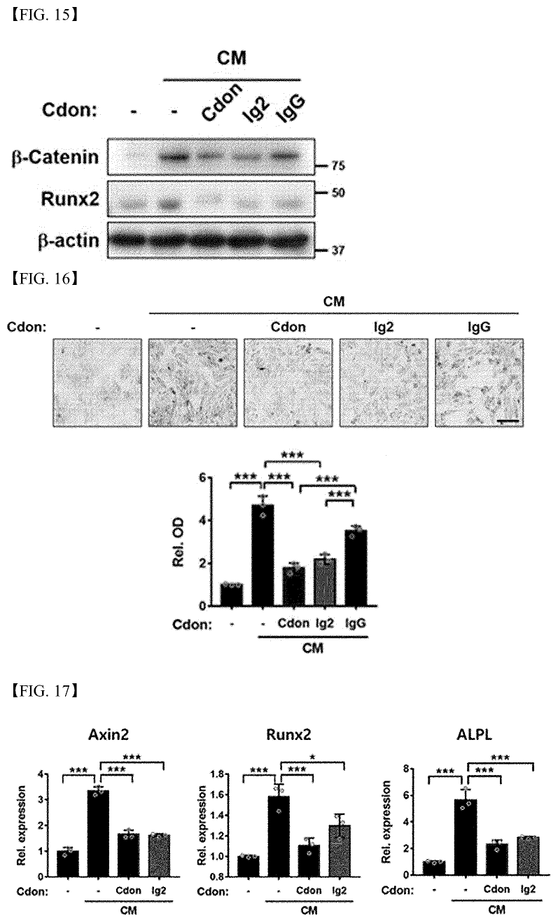

As a result, as shown in FIG. 15, when Cdon-Fc and Ig2-Fc were transfected, the expression of β-catenin and Runx2 was inhibited compared to the control group. In addition, as shown in FIG. 16, it was confirmed from the alizarin red staining results that the effect was not due to IgG, but rather due to Cdon and the Cdon-Ig2 domain. In addition, the expression levels of bone differentiation markers Runx2 and ALPL and the Wnt signaling factor Axin2 were analyzed to confirm that Cdon and the Cdon-Ig2 domain inhibited the induction of bone formation genes and the activation of the Wnt signaling pathway by a CM, as shown in FIG. 17. From the above-described results, it was confirmed that calcification in vascular cells may be reduced through the expression of the Cdon-Ig2 domain.

The present invention has been described with reference to preferred embodiments. Those skilled in the art will understand that the present invention may be implemented in modified forms without departing from the essential characteristics of the present invention. Therefore, the disclosed embodiments should be considered from an illustrative perspective rather than a restrictive perspective. The scope of the present invention is set forth in the claims, not in the foregoing description, and all differences within the scope equivalent thereto should be construed as being included in the present invention.

1. A method for preventing, ameliorating or treating vascular calcification or a vascular disease associated with vascular calcification, the method comprising administering to a subject in need thereof a Cdon-Ig2 domain or a peptide derived therefrom.

2. A method for preventing, ameliorating or treating vascular calcification or a vascular disease, the method comprising administering to a subject in need thereof a vector including a polynucleotide encoding a Cdon-Ig2 domain or a peptide derived therefrom, or a cell including the vector.

3. (canceled)

4. (canceled)

5. The method of claim 1, wherein the Cdon-Ig2 domain or the peptide derived therefrom includes a peptide including any one amino acid sequence selected from amino acid sequences of SEQ ID NOs: 1 to 4, or one or more amino acid sequences selected from amino acid sequences of SEQ ID NOs: 5 to 11.

6. The method of claim 1, wherein the disease associated with vascular calcification is selected from the group consisting of myocardial infarction, angiosclerosis (arteriosclerosis), hypertension, ischemic heart disease, perfusion disorders, cardiac hypertrophy, left ventricular hypertrophy, myocardial ischemia, peripheral arterial ischemia, and heart failure.

7. The method of claim 2, wherein the vector is linear DNA, plasmid DNA, or a recombinant viral vector.

8. The method of claim 7, wherein the recombinant viral vector is any one selected from the group consisting of a retrovirus, an adenovirus, an adeno-associated virus, a herpes simplex virus, and a lentivirus.

9. The method of claim 2, wherein the cell is any one selected from among a vascular muscle cell, a vascular smooth muscle cell, a vascular endothelial cell, an angioblast, or a vascular stem cell.

10. A method of providing information on vascular calcification or a vascular disease, comprising:
1) measuring the expression level of a Cdon-Ig2 domain or a peptide derived therefrom in a sample derived from a subject as an experimental group;
2) comparing the expression level of the Cdon-Ig2 domain or the peptide derived therefrom with the expression level of a Cdon-Ig2 domain or a peptide derived therefrom in a sample derived from a normal subject as a control group; and
3) determining that the risk of developing vascular calcification or a vascular disease associated therewith is high when the expression level of the Cdon-Ig2 domain or the peptide derived therefrom is lower compared to the control group.

11. The method of claim 10, wherein the expression level of the peptide in step 1) is detected by any one method selected from the group consisting of Western blotting, real-time polymerase chain reaction (qRT-PCR), enzyme-linked immunosorbent assay (ELISA), immunohistochemical staining, immunoprecipitation, and immunofluorescence.


SEQUENCE LISTING (12 sequences)

SEQ ID NO: 1 - protein, 1288 aa, synthetic construct
  REGION 1..1288: note = Human Cdon sequence
MHPDLGPLCTLLYVTLTILCSSVSSDLAPYFTSEPLSAVQKLGGPVVLHCSAQPVTTRISWLHNGKTLDGNLEHVIKIHQGTLTILSLNSSLLGYYQCLANNSIGAIVSGPATVSVAVLGDFGSSTKHVITAEEKSAGFIGCRVPESNPKAEVRYKIRGKWLEHSTENYLILPSGNLQILNVSLEDKGSYKCAAYNPVTHQLKVEPIGRKLLVSRPSSDDVHILHPTHSQALAVLSRSPVTLECVVSGVPAPQVYWLKDGQDIAPGSNWRRLYSHLATDSVDPADSGNYSCMAGNKSGDVKYVTYMVNVLEHASISKGLQDQIVSLGATVHFTCDVHGNPAPNCTWFHNAQPIHPSARHLTAGNGLKISGVTVEDVGMYQCVADNGIGFMHSTGRLEIENDGGFKPVIITAPVSAKVADGDFVTLSCNASGLPVPVIRWYDSHGLITSHPSQVLRSKSRKSQLSRPEGLNLEPVYFVLSQAGASSLHIQAVTQEHAGKYICEAANEHGTTQAEASLMVVPFETNTKAETVTLPDAAQNDDRSKRDGSETGLLSSFPVKVHPSAVESAPEKNASGISVPDAPIILSPPQTHTPDTYNLVWRAGKDGGLPINAYFVKYRKLDDGVGMLGSWHTVRVPGSENELHLAELEPSSLYEVLMVARSAAGEGQPAMLTFRTSKEKTASSKNTQASSPPVGIPKYPVVSEAANNNFGVVLTDSSRHSGVPEAPDRPTISTASETSVYVTWIPRANGGSPITAFKVEYKRMRTSNWLVAAEDIPPSKLSVEVRSLEPGSTYKFRVIAINHYGESFRSSASRPYQVVGFPNRFSSRPITGPHIAYTEAVSDTQIMLKWTYIPSSNNNTPIQGFYIYYRPTDSDNDSDYKRDVVEGSKQWHMIGHLQPETSYDIKMQCFNEGGESEFSNVMICETKVKRVPGASEYPVKDLSTPPNSLGSGGNVGPATSPARSSDMLYLIVGCVLGVMVLILMVFIAMCLWKNRQQNTIQKYDPPGYLYQGSDMNGQMVDYTTLSGASQINGNVHGGFLTNGGLSSGYSHLHHKVPNAVNGIVNGSLNGGLYSGHSNSLTRTHVDFEHPHHLVNGGGMYTAVPQIDPLECVNCRNCRNNNRCFTKTNSTFSSSPPPVVPVVAPYPQDGLEMKPLSHVKVPVCLTSAVPDCGQLPEESVKDNVEPVPTQRTCCQDIVNDVSSDGSEDPAEFSRGQEGMINLRIPDHLQLAKSCVWEGDSCAHSETEINIVSWNALILPPVPEGCAEKTMWSPPGIPLDSPTEVLQQPRET

SEQ ID NO: 2 - protein, 1256 aa, synthetic construct
  REGION 1..1256: note = Rat Cdon sequence
MHPDLGPLWKLLYVLVILCSSVSSDLAPYFISEPLSAVQKLGRPVVLHCSAKPVTARISWLHNGKRLDRNTEQIKIHRGTLTILSLNPSLSGCYQCVANNSVGAVVSGPATVSAAALADFDSSTMHVITAEEKNTGFIGCRVPESNPKAEVRYKIRGKWLMYSTGNYIILPSGNLQILNVSSKDKGSYKCAAYNPVTSELKVEPAGRKLLVSRPSSDGFHILHPALSQALAVLPHSPVTLECVVSGVPASQVYWLKDGQDALSGSNWRRLYSHLATASIDPADSGNYSCVVGNNRSGDVKHVTYTVNVLEHASISKGLHDQKVSLGATVRFTCEVHGNPAPNRTWFHNAQPIRPSSRHLTEGSVLKITGVIMEDSGLYQCMADNGIGFMQSTGRLQIEQDSGQRPVIVTAPANVEVTDGDFVTLSCNATGEPVPVIHWYGRHGLITSHPSQVLRSKSRKSHLFRPGDLDPEPVYLIMSQAGSSSLSIQAVTWEHAGKYTCEAVNKHGSTQSEAFLTVVPFETNTKAEPVTPSEASQNDERDPRDGSESGLLNLFPVKVHSGGVELPAEKNASVPDAPNILSPPQTHMPDTYTLVWRAGRDGGMPINAYFVKYRKLDDGSGAVGSWHTVRVPGSESELHLTELEPSSLYEVLMVARSAVGEGQPAMLTFRTSKEKMASSKNTQASFPPVGIPKRPVTSEASNSNFGVVLTDSSRHSGVPEAPDRPTISMASETSVYVTWIPRANGGSPITAFKVEYKRMKSSDWLVAAEDIPPSKLSVEVRSLEPGSIYKFRVIAINHYGESFRSSASRPYQVAGFPNRFSNRPITGPHIAYTEAVSDTQIMLKWTYIPSSNNNTPIQGFYIYYRPTDSDNDSDYKRDVVEGSKQWHTIGHLQPETSYDIKMQCFNEGGESEFSNVMICETKVKRVPGASEYPMKELSTPPSSSGNGGNVGPATSPARSSDMLYLIVGCVLGVMVLILLVFIALCLWKSRQQSAIQKYDPPGYLYQGSEINGQMVEYTTLSGTARINGSVHGGFLSKGSLSNGCSHLHHKGPNGVNGILNGTINGGLYSAHTSSLTRTCVEFEHPHHLVNGGAVYTAVPQMDPLECINCRNCRNNNRCFTKTNSPLPVVPVVASYPQDGLEMKPLGVMKFPVCPVSTVPDGGQIPEECLKDSVAPAPTQRTCRQDNTSDINSDSTEDTAEFNRGDSSGHSEAEDKVFSWSPLILSPVLEDCSEKTAWSPPGPPLDGLSVVLQQAQET

SEQ ID NO: 3 - protein, 94 aa, synthetic construct
  REGION 1..94: note = Human Cdon-ig2 domain sequence
ATVSVAVLGDFGSSTKHVITAEEKSAGFIGCRVPESNPKAEVRYKIRGKWLEHSTENYLILPSGNLQILNVSLEDKGSYKCAAYNPVTHQLKVE

SEQ ID NO: 4 - protein, 86 aa, synthetic construct
  REGION 1..86: note = Rat Cdon-ig2 domain sequence
TVSAAALADFDSSTMHVITAEEKNTGFIGCRVPESNPKAEVRYKIRGKWLMYSTGNYIILPSGNLQILNVSSKDKGSYKCAAYNPV

SEQ ID NO: 5 - protein, 4 aa, synthetic construct
  REGION 1..4: note = Cdon-Ig2 Ig strand A (120-123)
FDSS

SEQ ID NO: 6 - protein, 12 aa, synthetic construct
  REGION 1..12: note = Cdon-Ig2 Ig strand B (133-144)
KNTGFIGCRVPE

SEQ ID NO: 7 - protein, 7 aa, synthetic construct
  REGION 1..7: note = Cdon-Ig2 Ig strand C (149-155)
AEVRYKI

SEQ ID NO: 8 - protein, 4 aa, synthetic construct
  REGION 1..4: note = Cdon-Ig2 Ig strand D (157-160)
GKWL

SEQ ID NO: 9 - protein, 5 aa, synthetic construct
  REGION 1..5: note = Cdon-Ig2 Ig strand E (167-171)
YIILP

SEQ ID NO: 10 - protein, 7 aa, synthetic construct
  REGION 1..7: note = Cdon-Ig2 Ig strand F (173-179)
GNLQILN

SEQ ID NO: 11 - protein, 10 aa, synthetic construct
  REGION 1..10: note = Cdon-Ig2 Ig strand G (185-194)
KGSYKCAAYN

SEQ ID NO: 12 - protein, 125 aa, synthetic construct
  REGION 1..125: note = Rat Ig2 domain (artificial)
FDSSTMHVITAEEKNTGFIGCRVPESNPKAEVRYKIRGKWLMYSTGNYIILPSGNLQILNVSSKDKGSYKCAAYNPVKNTGFIGCRVPESNPKAEVRYKIRGKWLMYSTGNYIILPSGNLQILNV